(19) United States
(12) Patent Application Publication
(10) Pub. No.: US 2009/0305404 A1
(43) Pub. Date: Dec. 10, 2009

(54) Methods and compositions relating to blastomere-derived human embryonic stem cells

(75) Inventors: Ana Krtolica, San Francisco, CA (US); Dusko Ilic, San Francisco, CA (US)

Correspondence Address: WOLF GREENFIELD & SACKS, P.C., 600 ATLANTIC AVENUE, BOSTON, MA, 02210-2206, US

(73) Assignee: StemLifeLine, Inc., San Carlos, CA (US)

(21) Appl. No.: 12/455,954

(22) Filed: Jun. 9, 2009

Related U.S. Application Data
(60) Provisional application No. 61/131,561, filed on Jun. 9, 2008.
(60) Provisional application No. 61/196,984, filed on Oct. 22, 2008.

(51) Int. Cl. C12N 5/08

(52) U.S. Cl. 435/366

(57) ABSTRACT

The invention provides methods for producing human embryonic stem cells from blastomeres with reduced or no animal cells or products, including no serum regardless of source and including xeno-free conditions, without compromising derivation efficiency.

### RELATED APPLICATIONS

This application claims priority under 35 U.S.C §119(e) to U.S. Provisional Applications 61/131,561 and 61/196,984, filed on Jun. 9, 2008 and Oct. 22, 2008, respectively, the entire contents of all of which are incorporated by reference.

### FIELD OF THE INVENTION

The invention relates to the improved production of human embryonic stem cells from blastomeres.

### BACKGROUND OF THE INVENTION

The first human embryonic stem cells were derived from the inner cell mass of an embryo by Thomson et al. (Thomson et al. Science 282:1145-7, 1998.) Since that time, there has been a continuous effort to identify and develop derivation and culture conditions optimal for obtaining therapeutic grade stem cells suitable for future clinical applications. (Skottman et al. Regen. Med. 2:265-273, 2007.) Additional efforts have been aimed at producing embryonic stem cells without the destruction of an embryo, for ethical reasons. To this end, the ability to derive embryonic stem cells from single biopsied cells of a cleavage-stage embryo (i.e., blastomeres) without embryo loss has been investigated and shown possible. (Chung et al. Nature 439: 216-9, 2006; Klimanskaya et al. Nature 444: 481-5, 2006; Chung et al. Cell Stem Cell 2(2):113-117, 2008.)

The most efficient methods for embryonic stem cell production still have drawbacks however. For example, these methods still require the use of animal products, including animal feeder cells and animal serum. Exposure of human cells, particularly those intended for use in human subjects, to animal products may lead to the introduction of xenoantigens and more importantly xenopathogens into the human species.

### SUMMARY OF THE INVENTION

The invention relates in part to substantial and unexpected improvements in the methods for producing human embryonic stem cells from blastomeres. These improvements relate in part to the ability to produce human embryonic stem cells using reduced or no xenogeneic products. In particular, the methods of the invention are not dependent upon the use of murine feeder cells, nor are they dependent upon the continued use of animal serum throughout culture. Importantly, it has been found that animal serum if used at all can be restricted to a narrow window, and that the ultimately derived embryonic stem cells can be propagated in the final culture steps in the absence of such serum. These methods also are not dependent upon co-culture of blastomeres with the embryos from which they derive (i.e., the parental embryos) whether in the presence or absence of feeder cells. This allows the parental embryos to be cultured under conditions that are optimal for their development into blastocysts, thereby increasing the number of such parental embryos that can be used in vivo.

It was further found, according to a related aspect of the invention, that in some instances the derivation of embryonic stem cells from blastomeres in the presence of human feeder cells was particularly dependent upon the density of feeder cells used in culture. Thus, it was found that a particular range of feeder densities was important to optimal embryonic stem cell derivation. Some of the derivation methods described herein have an ES cell derivation efficiency of about 50%. All the derived lines maintained normal karyotype and expression of pluripotency markers for more than 20 passages and differentiated into all three germ layers (i.e., ectoderm, endoderm and mesoderm).

These and other improvements could not be anticipated or expected and were not predictable given the infancy of this area of stem cell research. Various combinations of the improvements provided by the invention result in an overall embryonic stem cell derivation efficiency approximating and in some cases exceeding that attained using the prior art optimal culture conditions involving mouse feeder cells and animal serum throughout the culture. Accordingly, the invention has unexpectedly and successfully replaced the prior gold standard of mouse feeder cells with human feeder cells, and it has reduced the dependency of the prior art methods on animal serum, without loss of efficiency or quality.

Thus, in one aspect, the invention provides a method for producing human embryonic stem cells comprising culturing a human blastomere and/or its progeny in the presence of human feeder cells, such as human adult feeder cells, and in the absence of other cells, such as other embryonic or fetal cells, for a time sufficient to generate embryonic stem cells, and isolating human embryonic stem cells.

In one embodiment, the human feeder cells are human foreskin fibroblast cells. In one embodiment, the human feeder cells are present in a density of about 2-3×10⁵ cells/ml.

In one embodiment, the human feeder cells are irradiated. In one embodiment, the human feeder cells are early passage feeder cells. In one embodiment, the human feeder cells have been passaged 4-8 times. In one embodiment, the human blastomere is not cultured in the presence of its parental embryo prior to culture with the human feeder cells. In one embodiment, the human embryonic stem cells are isolated at about 10-15 days of culture.

In one embodiment, the human blastomere is cultured in the absence of animal serum. In one embodiment, the human blastomere is cultured in the presence of animal serum for 4-10 days. In one embodiment, the human blastomere is cultured under xeno-free conditions. In one embodiment, the human blastomere is cultured in low oxygen.

In another aspect, the invention provides a method for improving the efficiency of human embryonic stem cell production from blastomeres comprising culturing a human blastomere and its progeny in the presence of human foreskin fibroblast cells and in the absence of other cells for a time sufficient to generate embryonic stem cells, wherein the human foreskin fibroblast cells are present at a density of about 2-3×10⁵ cells/ml, and isolating human embryonic stem cells.

In another aspect, the invention provides a method for improving the efficiency of human embryonic stem cell production from blastomeres comprising culturing a human blastomere and its progeny in the presence of human foreskin fibroblast cells and in the absence of other cells for a time sufficient to generate embryonic stem cells, wherein the human blastomere and human foreskin fibroblast cells are present in a ratio of about 1:10000 to about 1:15000, and isolating human embryonic stem cells.

In one embodiment, the human foreskin fibroblast cells are early passage feeder cells. In one embodiment, the human foreskin fibroblast cells have been passaged 4-8 times. In one embodiment, the human foreskin fibroblast cells are irradiated. In one embodiment, the human blastomere is not cultured in the presence of its parental embryo prior to culture with the human foreskin fibroblast cells. In one embodiment, the human embryonic stem cells are isolated at about 10-15 days of culture. In one embodiment, the human blastomere is cultured in the absence of animal serum. In one embodiment, the human blastomere is cultured in the presence of animal serum for 4-10 days. In one embodiment, the human blastomere is cultured in low oxygen. In one embodiment, the human embryonic stem cells are derived with at least a 25% efficiency. In one embodiment, the human embryonic stem cells are derived with at least a 30% efficiency. In one embodiment, the human embryonic stem cells are derived with about a 50% efficiency.

In another aspect, the invention provides a method for producing human embryonic stem cells comprising in a first culturing step culturing a human blastomere from a human embryo in the absence of feeder cells, in a second culturing step culturing the human blastomere and its progeny in the presence of human adult feeder cells and in the absence of other cells, in a third culturing step culturing the human blastomere and its progeny in the presence of human adult feeder cells and animal serum, in a fourth culturing step culturing the human blastomere and its progeny in the absence of animal serum, and isolating embryonic stem cells.

In one embodiment, the human blastomere is cultured with the human embryo in the first culturing step. In one embodiment, the first culturing step is about 12 hours in length.

In one embodiment, the human blastomere is cultured in the presence of human adult feeder cells and laminin, in the second culturing step. In one embodiment, the third and/or fourth culturing step occurs in the absence of laminin. In one embodiment, the human adult feeder cells are human foreskin fibroblast feeder cells. In one embodiment, the human blastomere and human adult feeder cells are seeded in about a 1:10000 to about a 1:15000 ratio in the second culturing step. In one embodiment, the human adult feeder cells are at a density of about 2-3×10⁵ cells/ml in the second culturing step. In one embodiment, the human adult feeder cells are irradiated. In one embodiment, the human adult feeder cells are early passage feeder cells. In one embodiment, the human adult feeder cells have been passaged at least 4-8 days. In one embodiment, the first, second, third and/or fourth culturing step is performed in low oxygen. In one embodiment, the third culturing step is 4-10 days in length. In one embodiment, the embryonic stem cells are isolated within 10-15 days after beginning the second culturing step.

These various methods provide ES cell derivation efficiencies of at least 25%, at least 30%, and in some instances about 50%.

In some embodiments, the ES cell lines are generated in the absence of serum and in the presence of low oxygen using human feeder cells that are human foreskin fibroblasts. The low oxygen level may range from about 2% to less than 20%, preferably from about 2% to about 15%, and more preferably from about 5% to about 10%. In one embodiment, the oxygen level is about 8%.

In still another aspect, the invention provides a method for producing human embryonic stem cells comprising culturing a human blastomere and/or its progeny in the presence of human foreskin fibroblasts, in the absence of other cells, in the absence of serum and in low oxygen for a time sufficient to generate embryonic stem cells, and isolating human embryonic stem cells. In one embodiment, low oxygen is 5-10% oxygen. In other embodiments, low oxygen is about 5%, about 6%, about 7%, about 8%, about 9%, or about 10% oxygen.

In another aspect, the invention provides a method for improving efficacy of human embryonic stem cell derivation in the absence of serum comprising culturing a human blastomere and/or its progeny in the absence of serum and in low oxygen for a time sufficient to generate embryonic stem cells, and isolating human embryonic stem cells. The method yields a derivation rate that is greater than the rate in the absence of serum and in normoxic conditions (i.e., about 20% oxygen). The difference in derivation rates may be 2-fold, 3-fold, 4-fold, 5-fold, or greater. In one embodiment, low oxygen is 5-10% oxygen. In other embodiments, low oxygen is about 5%, about 6%, about 7%, about 8%, about 9%, or about 10% oxygen. In another embodiment, low oxygen is about 8% oxygen. In some embodiments, the human blastomere and/or its progeny are cultured in the presence of human feeder cells. The human feeder cells may be human foreskin fibroblasts.

These and other embodiments of the invention will be described in greater detail herein.

Each of the limitations of the invention can encompass various embodiments of the invention. It is therefore anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways.

The phraseology and terminology used herein is for the purpose of description and should not be regarded as limiting. The use of “including”, “comprising”, or “having”, “containing”, “involving”, and variations thereof herein, is meant to encompass the items listed thereafter and equivalents thereof as well as additional items.

### BRIEF DESCRIPTION OF THE FIGURES

FIG. 1 is a series of photographs showing the derivation process including blastomere extraction and culture on human feeder cells, and the appearance of embryonic stem cell colonies.

FIG. 2A is a series of photographs showing the expression of a number of stem cell markers (i.e., SSEA-4, Oct-4, TRA-1-60, Nanog and TRA-1-81) in four embryonic stem cell lines generated according to the methods of the invention (i.e., W8-8A, W10-1A, W13-1C and W14-1A).

FIG. 2B is a series of photographs showing endogenous alkaline phosphatase activity in four embryonic stem cell lines generated according to the methods of the invention.

FIG. 2C is a series of photographs showing expression of endodermal (i.e., alpha-fetoprotein), mesodermal (i.e., smooth muscle actin) and ectodermal (i.e., beta III tubulin) differentiative potential from each of four embryonic stem cell lines generated according to the methods of the invention.

FIG. 3A is a series of photographs showing expression of a number of stem cell markers (i.e., alkaline phosphatase (AP) activity, Oct-4, Nanog, SSEA-4, TRA-1-60, and TRA-1-81) in an embryonic stem cell line generated according to the invention in the absence of serum using low oxygen. These results are representative of two other embryonic stem cell lines generated in an identical manner.

FIG. 3B is a series of photographs showing expression of ectodermal (i.e., beta III tubulin), mesodermal (i.e., smooth muscle actin), and endodermal (i.e., alpha feto protein) differentiative potential from an embryonic stem cell line generated according to the invention in the absence of serum using low oxygen. These results are representative of two other embryonic stem cell lines generated in an identical manner.

FIGS. 4A-F are a series of photographs that show expression in a newly generated hESC line using xeno-free conditions of pluripotency markers alkaline phosphatase (A), Nanog (B), Oct-4 (C), SSEA-4 (D), Tra 1-60 (E), and Tra 1-81 (F).

FIGS. 5A-C is a series of photographs showing expression of ectodermal marker beta III tubulin (A), mesodermal marker smooth muscle actin (B), and endodermal marker alpha-feto protein (C).

It is to be understood that the Figures are not required to enable the invention.

### DETAILED DESCRIPTION OF THE INVENTION

The invention relates to improved methods for producing embryonic stem cells from blastomeres. These improvements, in part, reduce or eliminate dependence of embryonic stem cell derivation and culture on animal products. Cellular therapeutics that do not contain and that have not been exposed to animal products are desirable for human clinical use. Much of the early stage work relating to the derivation and propagation of embryonic stem cells however has required murine feeder cells and bovine serum. The methods of the invention reduce or eliminate this dependence on animal products without any loss of derivation and propagation efficiency. The improvements described herein resulted in an overall average efficiency of ES cell derivation of about 29%. Certain specific culture methods described herein resulted in an efficiency of ES cell derivation of about 50%. Moreover, the methods derive embryonic stem cells from blastomeres without requisite loss of embryos, and therefore the ethical concerns relating to embryo use (and loss) are overcome.

The invention therefore provides in part xeno-free derivation of ES cells from human blastomeres. Xeno-free refers to conditions, and typically culture conditions, that lack components derived from non-human naturally occurring sources. These conditions may have synthetic components (i.e., components that are synthesized in vitro apart from any animal or other non-human cells or contamination).

The methods require harvest and culture of a blastomere from an early stage embryo. The embryos are preferably grade 1 or 2 cleavage stage embryos. They may be freshly prepared or frozen prior to blastomere extraction. The blastomeres (and their progeny) may be cultured in vitro culture for about 8-15 days or about 8-12 days. Initially, the blastomere is extracted from the embryo and cultured for about 12-24 hours in a first culture step. This culture step may occur in the presence of the embryo from which the blastomere was harvested (i.e., the parental embryo) but it may also occur in the absence of such embryo. One of the advantages of the methods described herein is the ability to culture the blastomere separately from its parental embryo, thereby allowing the parental embryo to be cultured under conditions that are optimal for its development.

The ES cell derivation methods described herein also do not require the presence of cells other than feeder cells and blastomeres and their progeny. In some important embodiments, the feeder cells are adult feeder cells and the cultures do not contain any embryonic or fetal cells apart from the blastomere and its progeny and, in some embodiments, the intact parental embryo. The only cells within the cultures therefore may be the blastomere (and its progeny) and the feeder cells and, in some embodiments, the parental embryo. The parental embryo, if present, is generally maintained intact. Such cultures are referred to as lacking other cells (or being performed in the absence of other cells) meaning that the cultures do not include cells that are not the feeder cells, not the parental embryo, and not the blastomere or its progeny.

The initial culture of the extracted blastomere therefore does not require the presence of other embryonic or fetal cells. It also does not require feeder cells, nor does it require the parental embryo. This culture step employs any medium that supports the growth of an embryo (referred to herein as “embryo medium”). An example of a suitable embryo medium is cleavage medium such as but not limited to Quinn's Advantage™ Protein Plus Cleavage Medium, commercially available from Sage, Cat. No. 1526, optionally including 10 μg/ml human laminin, commercially available from Sigma, Cat. No. L6274.

This and other culture steps described herein preferably are performed in a small and contained volume due to the single or low cell number nature. Thus, it can be performed in a microwell of a multiwell plate, or it may be performed in a confined droplet of media. The volume of such drops may vary and, in some embodiments, may range from about 25-60 μl.

During this first culture step, a proportion of the blastomeres divide to produce at least two progeny. The originally extracted cells and their progeny are referred to herein collectively as blastomeres.

The first culture (or culturing) step is followed by a second culture step in which the blastomeres from the first culture step are cultured in the presence of human feeder cells. If the blastomeres were cultured with parental embryos in the first culture step, such parental embryos are removed before or at this step and may be cultured separately prior to optional freezing. The media used in the second culture step is preferably changed to a blastocyst media (e.g., Quinn's Advantage™ Protein Plus Blastocyst Medium, commercially available from Sage, Cat. No. 1529), and it may include laminin (e.g., 10 μg/ml human laminin, commercially available from Sigma, Cat. No. L6274), basic FGF (e.g., 25 ng/ml basic FGF from R&D, Cat. No. 233-FR), and/or LIF (e.g., 10 ng/ml). Other extracellular matrix proteins such as fibronectin may be used in various embodiments, but are not required.

As discussed above, this culture step is also preferably performed in a small or contained volume. As an example, it may be performed in microliter-sized drops of media (e.g., 25-60 μl). The Examples demonstrate the use of 50 μl drops and particular feeder cell and blastomere seeding densities. Given the small volume of these culture steps and the importance of feeder cell density, it is important to prevent evaporation of the media during the culture period. One way to achieve this is to cover drops with oil, thereby containing the media and preventing its evaporation.

During this culture period, blastomere attachment is generally observed within about 24-72 hours. Starting at about day 4 of co-culture with feeder cells, a fraction (e.g., 20-80%) of the media volume is removed and replaced on a daily basis. This second culturing step continues for about five days from the time of initial co-culture of blastomeres with feeder cells. This is generally the time at which cellular outgrowths are observed in the culture. The first outgrowths observed are trophectoderm-like outgrowths. These cells initially appear as a tightly packed monolayer of large relatively flat cells, however within about 1-2 days, these cells are observed to “round up” and form clumps on feeder-denuded regions of the solid support, at which point they may be removed mechanically and/or by virtue of regular media changes. By about day 7, most of these trophectoderm outgrowths are removed from the culture.

At about the initial observation of these first outgrowths, the blastocyst media is changed to embryonic stem cell media which may optionally include fetal bovine (or calf) serum. An example of ES cell medium comprises 80% KnockOut™ DMEM, 20% KnockOut™ SR, 1% non-essential amino acids, and 2 mM L-glutamine, all commercially available from Gibco/Invitrogen under Cat. Nos. 10829, 10828, 11140, and 25030, and 10 μM beta-mercaptoethanol commercially available from Sigma, Cat. No. M7522. This media change reflects the start of the third culturing step. This culture step does not require the presence of laminin. At about day 6-9 (from the initial culturing on the human feeder cells) compact uniform clusters of cells, resembling embryonic stem cells colonies, begin to form. These clusters are allowed to divide until they contain approximately about 200-300 cells (as determined by visual inspection), after which they are mechanically disrupted into smaller clusters which are left in the same drop to continue growth. These smaller clusters re-attach to the feeder cells within about 24 hours after disruption and then form embryonic stem cell colonies within 2-3 days. These colonies are mechanically dissected and transferred to a larger volume co-culture with the feeder cells. At this point, serum is removed from the culture. This latter step represents the fourth culturing step. At this point and beyond, the cells may be propagated under serum-free conditions. Thus, when serum is used, the blastomeres and their progeny may be cultured in serum for 4-10 days in some instances.

Various aspects of the foregoing culture method are discussed in greater detail below, including in the Examples.

The method may be carried out using embryos from any source. The most common source of embryos is in vitro fertilization (IVF) clinics. The highest quality or grade embryos are preferable. The Examples describe the use of grade 1 or 2 cleavage stage embryos, for example. The invention contemplates the use of surplus embryos from IVF procedures for fertility purposes, as well as embryos that are generated particularly for stem cell line generation. The invention also contemplates the use of embryos produced by somatic cell nuclear transfer, parthenogenesis, androgenesis or other asexual techniques. Embryos derived from sexual reproduction may be referred to herein as “fertilized embryos” in order to distinguish them from asexually derived embryos.

The embryos used in the methods of the invention can be freshly prepared or they may be previously cryopreserved. If cryopreserved, the embryos are thawed out according to methods known in the art including those generally used in IVF clinics. As an example, the embryos may be thawed using an embryo thawing kit (Cooper Surgical, ART8016). Embryos may be thawed at room temperature in air for about 2 minutes, followed by incubation at 37° C. for 3 minutes, and then incubation for 10 minutes in 0.5 M sucrose thawing medium. The embryos are then transferred to 0.3 M sucrose thawing medium and incubated for 10 minutes, after which they are washed several times and then placed into embryo medium (e.g., cleavage medium).

The thawed embryos are then incubated in cleavage medium, optionally under oil, for a short period of time (e.g., 1-3 hours). The cleavage medium may be but is not limited to Quinn's cleavage medium (e.g., Quinn's Advantage™ Protein Plus Cleavage Medium, commercially available from Sage, Cat. No. 1526), optionally with human laminin (e.g., 10 μg/ml). Prior to extraction, embryos may be incubated in the presence of medium containing polyvinyl alcohol (PVA) in order to loosen cell-cell interactions and thereby facilitate zona pellucida opening and blastomere extraction. Alternatively, the embryo may be incubated in calcium, magnesium-free medium.

Blastomeres may be extracted from embryos at this point, or alternatively, the embryos may be cultured for 24-48 hours prior to blastomere extraction. Generally, blastomeres are extracted from embryos having 5-12 cells (i.e., blastomeres), or those having 8-12 cells, or those having 10-12 cells. Preferably only one blastomere is extracted from the embryo although, in some instances, two blastomeres may be extracted without any noticeable effect on the development of the parental embryo. In these latter instances, both blastomeres may be used to establish an embryonic stem cell line, or alternatively one may be used for genetic screening of the resultant line as well as the parent embryo.

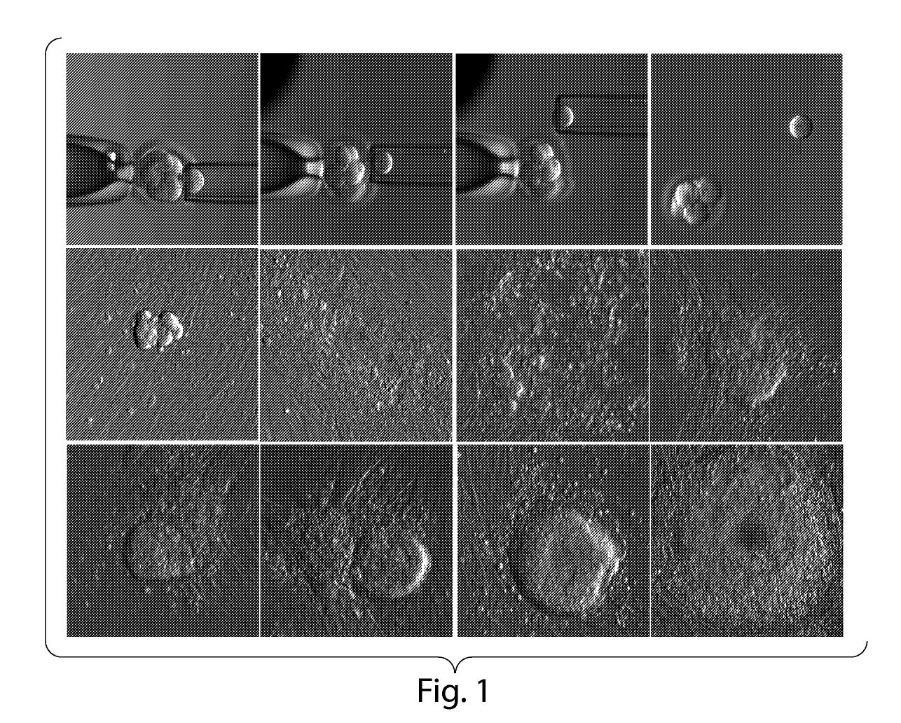

The prior art teaches various methods for blastomere extraction from embryos. These methods first require opening of the zona pellucida. This can be accomplished through physical, chemical or enzymatic methods. The Examples described herein open the zona pellucida using a non-contact laser while holding the embryo fixed using for example a micropipette. FIG. 1 demonstrates such an arrangement. Examples of physical methods include partial dissection of the zona pellucida using a micropipette and drilling of the zona pellucida using a piezo drill. An example of a chemical method is the partial digestion of the zona pellucida using Tyrode acid. An example of an enzymatic method is the partial digestion of the zona pellucida with pronase or other proteases.

Once the zona pellucida is opened, blastomeres may be removed using biopsy micropipettes and micromanipulators and by applying mild suction to the embryo.

The base media used to incubate and/or culture blastomeres and/or embryonic stem cells are generally those used in the art and include cleavage media (such as but not limited to Quinn's cleavage media, commercially available from Sage (as described above) or Cooper Surgical Inc.), blastocyst media (such as but not limited to Quinn's blastocyst media, commercially available from Sage (as described above) or Cooper Surgical Inc.), and embryonic stem cell media (such as 80% KnockOut™ DMEM from Gibco/Invitrogen, Cat. No. 10829 which includes 4.5 g/L D-glucose, sodium pyruvate, and no L-glutamine), 20% KnockOut™ SR from Gibco/Invitrogen, Cat. No. 10829, 1% non-essential amino acids from Gibco/Invitrogen, Cat. No. 11140, 2 mM L-glutamine from Gibco/Invitrogen, Cat. No. 25030, and 10 μM beta-mercaptoethanol from Sigma, Cat. No. M7522), and optionally basic fibroblast growth factor, as well as other media commercially available from Millipore and Invitrogen). Further restrictions and/or supplements to these media are as described herein. In some embodiments, LIF is added to the culture medium.

Preferably the human feeder cells are human foreskin fibroblast feeder cells. However, in some instances the invention contemplates the use of other human feeder cells, including other adult feeder cells. The human foreskin fibroblast feeder cells, for purposes of the invention, are considered to be adult feeder cells rather than embryonic or fetal feeder cells. Such cells preferably are screened for pathogens prior to use. The feeder cells and the blastomeres are non-autologous in some embodiments.

Other examples of human feeder cells which may be used in some embodiments of the invention include oral fibroblasts, skin fibroblasts, oviduct fibroblasts, breast fibroblasts (e.g., such as those harvested during reduction mammoplasty), endometrial fibroblasts or epithelial cells, endometrial stromal cells, fallopian tube fibroblasts, placental fibroblasts, amniotic epithelial cells (preferably harvested at term), granulosa cells (preferably harvested after oocyte retrieval), lung fibroblasts and other tissue derived stromal cells or fibroblasts.

The feeder cells are harvested and grown in culture in order to generate large numbers of these cells. It has been discovered according to the invention that the efficiency of embryonic stem cell derivation is improved when early passage feeder cells are used compared to later passage feeder cells. As used herein, early passage feeder cells are feeder cells that have been passaged up to 10 times, while later passage feeder cells have been passages more than 10 times. Passage of cells refers to the process whereby cells are harvested from their existing culture (usually using enzymatic methods), and then used to seed a larger culture. Passages may be a one in two split, meaning that the cells from one culture vessel are harvested and used to seed two culture vessels of the same surface area. One in three, one in four, one in five, or more dilute splits are contemplated by the invention. The feeder cells may have been passaged 1 time, 2 times, 3 times, 4 times, 5 times, 6 times, 7 times, 8 times, 9 times or 10 times. In some instances, they have been passaged 4-10 times, 6-10 times, 4-8 times, or 6-8 times. Feeder cells that have been passaged more than 10 times may also be used in the methods of the invention, although ES cell derivation efficiency may be reduced.

Once a sufficient number have been grown by repeated culturing and splitting, the cells are optionally mitotically inactivated and stored for later use. Mitotic inactivation means that the cells are treated in a manner that prevents them from dividing further but that is not cytotoxic to the majority of the cells. Thus the cells can continue to produce factors necessary for stem cell generation and maintenance even though they are incapable of cell division. Before or after being mitotically inactivated, the feeder cells can be cryopreserved (frozen) for future use in appropriate aliquots. Mitotic inactivation of feeder cells can be accomplished by ultraviolet (UV), X-, or gamma-irradiation (e.g., at 10-50 Gy), or by chemical means such as senescence inducing drugs (e.g., mitomycin C, toyocamycin, tertbutylhydroperoxide (t-BHP) and hydrogen peroxide (H₂O₂)). As an example, the feeder cells may be inactivated by exposure to 50 Gy of gamma-irradiation.

In important embodiments, once the blastomeres are transferred into a culture volume (e.g., a drop) that contains human feeder cells, no other cells are required. As an example, the parental embryo, cells from the parental embryo, and/or other embryonic or fetal cells (apart from the progeny of the blastomere itself) are not required and are not present in the culture. As used herein, a blastomere/feeder cell co-culture excludes the presence of adult, fetal or embryonic cells that are not the feeder cells, the blastomeres, or cells that are endogenously produced in the culture as a result of division and/or differentiation of the blastomeres. Thus, in some important aspects of the invention, the second culturing step is a blastomere/feeder cell co-culture and it contains the feeder cells, and the blastomeres and their progeny, but no other cells.

In accordance with the invention, the derivation method is particular dependent upon the density of human feeder cells used in the second culturing step. In a preferred embodiment, the human feeder cells are introduced into the media drops and then allowed to settle and grow for a period of time. The number of cells will depend on the volume of the drop. It has been found according to the invention that a narrow range of feeder densities are optimal for embryonic stem cell derivation efficiency. The invention contemplates in various embodiments, feeder densities that range from about 2-4×10⁵ cells/ml, or from about 2.25-3.75×10⁵ cells/ml, or from about 2.5-3.5×10⁵ cells/ml. In one embodiment, the feeder cell density is about 2-3×10⁵ cells/ml. This is achieved by placing about 10000-15000 human feeder cells into a drop having a volume of about 50 μl. These feeder cells are then allowed to attach and grow (but not divide due to inactivation) for a few days (e.g., 3-4 days), after which time blastomeres are introduced into the drops.

The methods may include, in some instances, the use of animal serum such as fetal bovine (or calf) serum (FBS or FCS), although they are not dependent upon such use. Thus, in some instances, the methods are referred to as animal serum free methods, meaning that at no point in the culturing steps is an animal serum exogenously added. If used, however, animal serum such as FCS is introduced after blastomeres are co-cultured with the feeder cells and initial outgrowths are observed. The total period of time in which the blastomeres are exposed to animal serum may be less than 10 days, less than 9 days, less than 8 days, less than 7 days, less than 6 days, less than 5 days, less than 4 days, less than 3 days, or less than 2 days. In some embodiments, blastomeres (including their progeny) are cultured in the presence of animal serum for about 4-10 days, about 5-9 days, or about 6-8 days. In still other embodiments, the exposure to animal serum may be as short as 2-4 days, or 2-3 days. In some embodiments, blastomeres and their progeny are exposed to animal serum between days 6 or 7 through to days 9 or 10 of culture in the presence of feeder cells.

The amount of serum may be 20% or 15% but is more preferably 10% or lower, including 9%, 8%, 7%, 6%, 5%, 4%, 3%, 2% or 1% (volume by volume), or less. Suitable animal serum is commercially available from a number of sources including Hyclone (e.g., FBS). In some embodiments, all of the culturing steps are performed in the absence of animal serum. In other embodiments, all the culturing steps are performed in the absence of human serum. In important embodiments, all the culturing step are performed in the absence of serum, regardless of its source.

In some embodiments of the invention, the hESC are generated using a low oxygen or hypoxic condition or environment. A low oxygen or hypoxic condition is a culture condition having at least 2% but less than 20% oxygen content. Depending on the embodiment, oxygen content may be equal to or less than 15%, equal to or less than 10%, equal to or less than 9%, equal to or less than 8%, equal to or less than 7%, equal to or less than 6%, equal to or less than 5%, equal to or less than 4%, or equal to or less than 3%, provided that it is equal to or greater than 2%. In some embodiments, the oxygen content is 5-10%. In some embodiments, oxygen content is about 5%, about 6%, about 7%, about 8%, about 9%, or about 10%.

In some important embodiments, the hESC are generated in the absence of serum at low oxygen and in the presence of feeder cells such as human foreskin fibroblasts.

Human embryonic stem cell colonies have a distinctive morphology and their presence in the cultures of the invention can be deduced by visual examination. Generally, these colonies are adherent (e.g., to the underlying feeder monolayer) and compact.

As used herein, human embryonic stem cells are cells derived from human embryos that are pluripotent but not totipotent (i.e., they are able to generate many or most human tissues but they are not able to generate another individual). The human embryonic stem cells generated according to the methods of the invention therefore can be used to generate one or more specific cell lineage(s) or tissue(s) but not an entire organism. Characteristics of human embryonic stem cells include high nucleus to cytoplasm ratio, prominent nucleoli and the ability to form compact colonies in vitro, expression of markers such as alkaline phosphatase, stage-specific embryonic antigens (SSEA) 3 and 4, TRA-1-60 and TRA-1-81, a normal karyotype (i.e., 22 pairs of autosomal chromosomes and a pair of sex chromosomes, for a total of 46 chromosomes), the ability to develop into one or more mesodermal lineages (e.g., bone, cartilage, smooth muscle, striated muscle and hematopoietic cells), one or more endodermal lineages (e.g., liver, primitive gut and respiratory epithelium), and one or more ectodermal lineages (e.g., neurons, glial cells, hair follicles and tooth buds), immortality as defined by the ability to exist in culture for extended periods of time (e.g., many months, up to a year or more, potentially indefinitely, without differentiating completely and without exhaustion) and/or expression of telomerase activity and the ability to maintain telomere length.

As used herein, a human embryonic stem cell line is an isolated population of embryonic stem cells derived in vitro from a single embryo. Each line may be regarded as a monoclonal line since it was generated from a single embryo. The line may comprise differentiated progeny of the embryonic stem cells.

The invention provides for future use of the generated human embryonic stem cell lines. Such use may occur within months or years after the establishment of the cell line. The stem cell lines therefore may be stored indefinitely such as by cryopreservation. Methods for cryopreserving embryonic stem cell lines are known in the art and have been described in Ji et al. Biotechnol. Bioeng. 2004, 5:299-312; Richards et al., Stem Cells 2004, 22:779-789; Reubinoff et al. Human Reprod. 2001, 10:2182194. It is to be understood however that in some embodiments the stem cell line may be used prior to cryopreservation, and directly from culture. The invention is not limited in this manner.

The invention contemplates that the human embryonic stem cells produced according to the methods described herein may be used autologously. This is because the parental embryo from which the hESC are derived is fully capable of generating a human subject, and that human subject is genetically identical to the hESC generated. In this respect, the hESC are considered “custom” or “customized” cells (and lines) for the person to whom they are autologous. The invention however also contemplates that such hESC may be used clinically for other individuals.

The hESC can be used in both research and therapeutic purposes. They can be differentiated into a number of lineages including but not limited to endothelial cells, neurons, hematopoietic cells, cardiomyocytes, skeletal muscles, hepatocytes, insulin-producing cells, glial progenitor cells, osteoblasts, gametes and kidney cells. Accordingly, they can be used in a transplant setting in the treatment (including prevention) of various conditions including but not limited to Parkinson's disease (dopaminergic neurons), Alzheimer's disease (neural precursors), Huntington's disease (GABAergic neurons), blood disorders such as leukemia, lymphoma myeloma and anemia (hematopoietic cells), side-effects of radiation e.g., in transplant patients (hematopoietic precursors), myocardial infarction, ischemic cardiac tissue or heart-failure (partially- or fully-differentiated cardiomyocytes), muscular dystrophy (skeletal muscle cells), liver cirrhosis or failure (hepatocytes), chronic hepatitis (hepatocytes), diabetes including type I diabetes (insulin-producing cells such as islet cells), ischemic brain damage (neurons), spinal cord injury (glial progenitor cells and motor neurons), amyotrophic lateral sclerosis (ALS) (motor neurons), orthopedic tissue injury (osteoblasts), kidney disease (kidney cells), corneal scarring (corneal stem cells), cartilage damage (chondrocytes), bone damage (osteogenic cells including osteocytes), osteoarthritis (chondrocytes), myelination disorders such as Pelizaeus-Merzbacher disease, multiple sclerosis, adenoleukodystrophies, neuritis and neuropathies (oligodendrocytes), and hair loss. References documenting the differentiation of embryonic stem cells into these various lineages include Bjorklund et al., 2002, PNAS USA 99:2344-2349 (dopaminergic neurons), West and Daley, 2004, Curr Opin Cell Biol 16:688-692; U.S. Pat. No. 6,534,052 B1; Kehat and Gepstein, 2003, 8:229-236; Nir et al., 2003, 58:313-323; U.S. Pat. Nos. 6,613,568 and 6,833,269. In vitro as well as in vivo differentiation is contemplated by the invention. Thus, transplant of differentiated cells and/or undifferentiated or partly differentiated embryonic stem cells is embraced by the invention.

It is to be understood that one of the benefits provided by some of the methods of the invention is the avoidance of contamination of the hESC with xeno-pathogens or xeno-antigens. Thus, the methods may avoid or preclude various levels of testing of the hESC prior to in vivo use. In some embodiments, it is anticipated that the hESC and/or their differentiated progeny can be transplanted into an individual without prior testing for xeno-pathogen content.

The invention also contemplates the ability to transduce embryonic stem cells or their differentiated progeny with particular nucleic acids prior to transplant.

The invention provides yet another use for the human embryonic stem cells generated according to the methods described herein. The hESC can be used to screen various agents for toxicity and in some embodiments therapeutic efficacy. The readouts from such in vitro assays are correlative of the in vivo toxicity or efficacy such agents would exhibit in human subjects that are autologous to the hESC. Thus, the effect of the agent on the differentiated embryonic stem cells in vitro is a form of surrogate marker or readout for how the agent will function in vivo in the human subject. Using this technique it should be possible to customize a therapy for an individual by identifying agents that are safe and efficacious from those that are not.

In one instance, the hESC are differentiated into one or more particular cell lineages and those differentiated progeny are then exposed to the agent. As described herein, it is now possible to differentiate embryonic stem cells into various cell lineages including but not limited to melanocytes, hematopoietic cells, hepatocytes, kidney cells, skeletal muscle cells, dopaminergic neurons, glial cells, cardiomyocytes, endothelial cells, and osteoblasts. Thus for example a subject that has or is at risk of developing leukemia would want to screen differentiated hematopoietic cells for their response profile to one or more anti-leukemia agents. As another example, a subject having muscular dystrophy would want to screen differentiated skeletal muscle cells for their response to one or more agents intended for use in muscular dystrophy.

The agents to be tested include those used clinically as well as experimental agents.

In some more common embodiments, such testing will focus on the cytotoxicity of drugs in particular differentiated lineages from the embryonic stem cells. Accordingly, in these assays, the readout would be cell death (or conversely cell viability).

Drugs that can be tested according to these methods particularly for whether they are toxic to cells of a particular genetic background include but are not limited to adrenergic agent; adrenocortical steroid; adrenocortical suppressant; aldosterone antagonist; anabolic; analeptic; analgesic; androgen; anesthesia, adjunct to; anesthetic; anorectic; anterior pituitary suppressant; anti-acne agent; anti-adrenergic; anti-allergic; anti-androgen; anti-anemic; anti-anginal; anti-arthritic; anti-asthmatic; anti-atherosclerotic; anticholelithic; anticholelithogenic; anticholinergic; anticoagulant; anticoccidal; anticonvulsant; antidepressant; antidiabetic; antidiarrheal; antidiuretic; anti-emetic; anti-epileptic; anti-estrogen; antifibrinolytic; antiglaucoma agent; antihemophilic; antihemorrhagic; antihistamine; antihyperlipidemia; antihyperlipoproteinemic; antihypertensive; antihypotensive; anti-inflammatory; antikeratinizing agent; antimigraine; antimitotic; antimycotic, antinauseant, antineoplastic, antineutropenic, antiparkinsonian; antiperistaltic, antipneumocystic; antiproliferative; antiprostatic hypertrophy; antipruritic; antipsychotic; antirheumatic; antiseborrheic; antisecretory; antispasmodic; antithrombotic; antitussive; anti-ulcerative; anti-urolithic; benign prostatic hyperplasia therapy agent; blood glucose regulator; bone resorption inhibitor; bronchodilator; carbonic anhydrase inhibitor; cardiac depressant; cardioprotectant; cardiotonic; cardiovascular agent; choleretic; cholinergic; cholinergic agonist; cholinesterase deactivator; coccidiostat; cognition adjuvant; cognition enhancer; depressant; diagnostic aid; diuretic; dopaminergic agent; ectoparasiticide; emetic; enzyme inhibitor; estrogen; fibrinolytic; free oxygen radical scavenger; gastrointestinal motility effector; glucocorticoid; gonad-stimulating principle; hair growth stimulant; hemostatic; histamine H2 receptor antagonists; hormone; hypocholesterolemic; hypoglycemic; hypolipidemic; hypotensive; immunomodulator; immunoregulator; immunostimulant; immunosuppressant; impotence therapy adjunct; keratolytic; LHRH agonist; liver disorder treatment; luteolysin; mental performance enhancer; mood regulator; mucolytic; mucosal protective agent; mydriatic; nasal decongestant; neuromuscular blocking agent; neuroprotective; NMDA antagonist; non-hormonal sterol derivative; oxytocic; plasminogen activator; platelet activating factor antagonist; platelet aggregation inhibitor; post-stroke and post-head trauma treatment; progestin; prostaglandin; prostate growth inhibitor; prothyrotropin; psychotropic; pulmonary surface; relaxant; repartitioning agent; scabicide; sclerosing agent; sedative; sedative-hypnotic; selective adenosine A1 antagonist; serotonin antagonist; serotonin inhibitor; serotonin receptor antagonist; steroid; symptomatic multiple sclerosis; thyroid hormone; thyroid inhibitor; thyromimetic; tranquilizer; amyotrophic lateral sclerosis agent; cerebral ischemia agent; Paget's disease agent; unstable angina agent; uricosuric; vasoconstrictor; vasodilator; wound healing agent; xanthine oxidase inhibitor. Those of ordinary skill in the art will know or be able to identify agents that fall within any of these categories, particularly with reference to the Physician's Desk Reference.

The present invention is further illustrated by the following Examples, which in no way should be construed as further limiting.

### EXAMPLES

### Example 1

### Human Embryonic Stem Cell Lines from Biopsied Blastomeres with Minimal Exposure to Xenomaterials

### Summary:

A protocol was established for derivation and culturing of human embryonic stem cells (hESC) from biopsied blastomeres with minimal exposure to materials of animal origin. Blastomeres are isolated mechanically without jeopardizing embryo development. Cell lines are derived and propagated in defined medium on feeders of human origin. Presence of fetal calf serum is required only during initial outgrowth formation. This method allows generation of clinical-grade hESC while preserving parental embryos.

### Materials and Methods:

Supernumerary cleavage stage embryos generated for clinical purpose were obtained from in vitro fertilization (IVF) clinics with full informed consent and used in compliance with institutional review board standards. 29 grade I or II embryos were thawed and incubated for 3 hours in Quinn's cleavage medium under oil. Prior to micromanipulation, embryos were incubated with PVA (0.05% PVA) for 10-15 minutes to loosen cell-cell interactions. Embryos were held in place by gentle suction using a holding micropipette in a way that placed blastomere to be biopsied at the 3:00 o'clock position (FIG. 1). An opening in the zona pellucida was created using a non-contact laser and blastomeres were removed by applying mild suctions using biopsy micropipette and micromanipulator. Parental embryos and biopsied blastomeres were cultured together for the next 24 h.

Embryos were transferred then to Quinn's blastocyst medium where they continued development. Twenty four to 48 h later, when they reached blastocyst stage, embryos were cryopreserved. Some embryos were cultured in the absence of their parental embryo.

After 24 hours of this initial culture (whether in the presence or absence of the parental embryo), 65.5% of blastomeres divided at least once. All blastomeres were transferred onto irradiated human foreskin fibroblast (HFF) feeder layers in 50 μl drops of Quinn's blastocyst medium supplemented with 10 μg/ml laminin. Drops were covered with oil to prevent evaporation. The majority (94.7%) of divided blastomeres attached within 24-72 h after plating on HFF feeders. After the blastomeres were attached, ½ of the volume in each drop was replaced with a fresh medium on a daily basis. 77.8% of attached blastomeres produced outgrowth within the next 5 days. After five days on HFF, Quinn's blastocyst medium was switched to hESC derivation medium (containing 10% FBS). Laminin was omitted from the medium once the initial outgrowth was observed. Initial outgrowth on HFF contained cells of various morphologies.

Trophectoderm-like cells generally dominated the culture between days 5-6. Initially, they appeared as a tightly packed monolayer of large relatively flat cells. At days 6-7, the cells were rounding up and forming clumps within feeder-denuded areas. Clumps were then removed in the process of media changes. Between days 9 and 12, 4/14 (or 28.6%) of cultures contained compact uniform clusters of cells resembling hESC colonies. When the initial hESC-like colonies reached a size of about 200-300 cells per colony, they were mechanically dissected into smaller pieces and left in the same drop. Over 90% of dissected pieces re-attached within 24 h and formed hESC-like colonies in 2-3 days. The colonies were again mechanically dissected and transferred into one well of 4-well dish with HFF. From that point forward, the cells were continuously expanded and characterized under serum-free condition in defined medium.

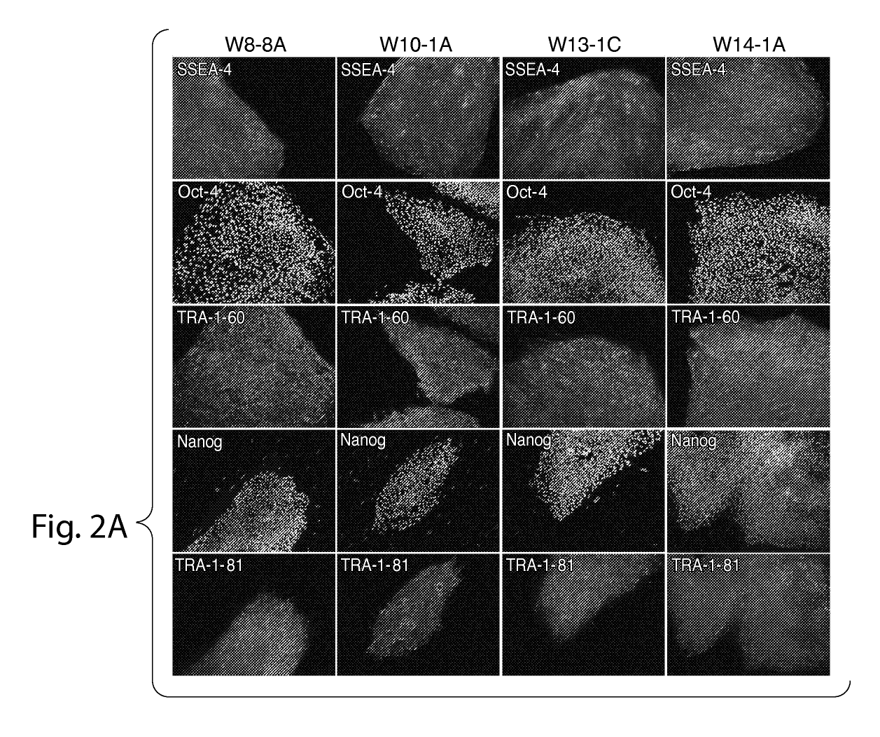

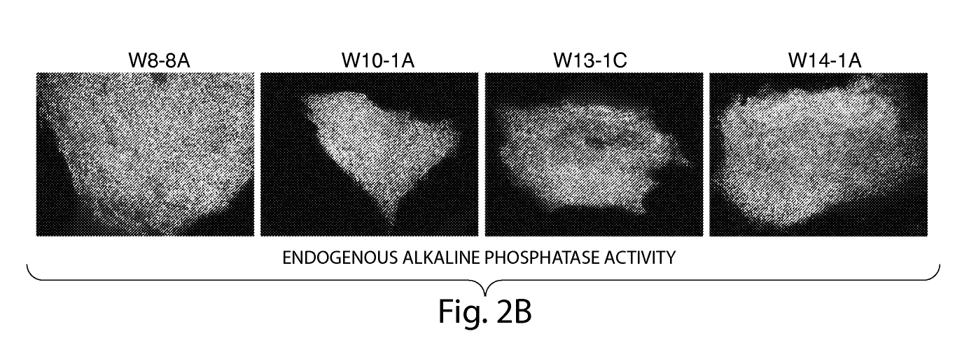

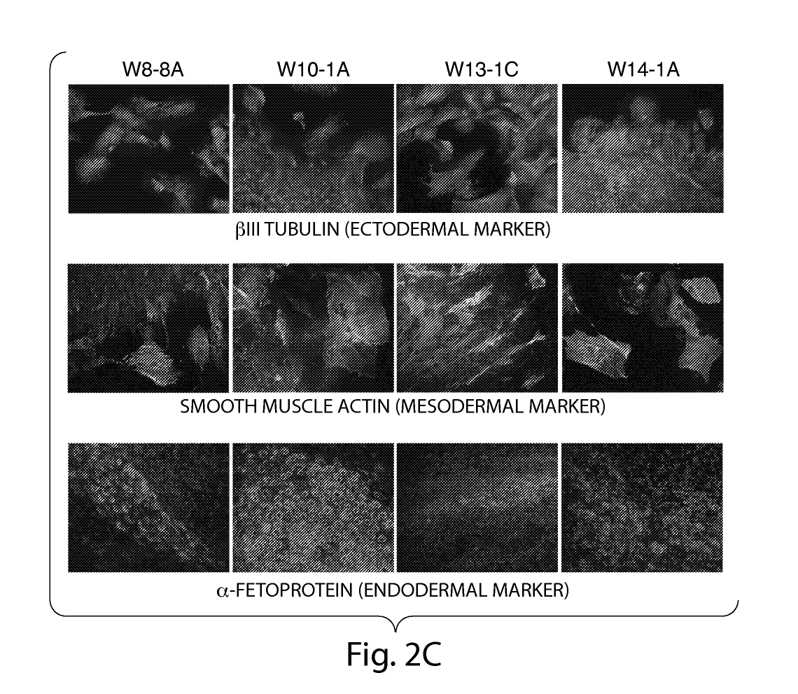

Four lines, W8-8A, W10-1A, W14-1A and W13-1C, were generated. All four lines showed normal karyotype. Lines W8-8A, W10-1A, and W14-1A had a 46,XX karyotype, while line W13-1C had a 46,XY karyotype. All four lines expressed molecular markers of pluripotency (FIGS. 2A and B). Upon differentiation in culture all four lines expressed markers of all three germ layers (FIG. 2C) confirming their pluripotent potential.

### Example 2

### Improvement of hESC Derivation Efficacy on Human Foreskin Fibroblasts

Human foreskin fibroblasts (HFF) are reported to support hESC derivation using passage numbers (PD) 9-25 (Amit et al. Biol Reprod. 68(6):2150-6, 2003; Hovatta et al. Human Reprod. 18(7):1404-1409, 2003). A set of experiments was conducted in which hESC derivation efficacy from a genetically identical source was compared on feeder cells that had been passaged 14 times (i.e., PD14) versus feeder cells that had been passaged 6 times (i.e., PD6).

Six grade 1 and 2 surplus frozen human cleavage stage embryos donated by two different consenting couples were thawed and incubated in Quinn's cleavage medium for a minimum of 3 hours at standard culture conditions. To obtain genetically identical samples for comparison, two blastomere were extracted from each embryo. About ⅔ of biopsied blastomeres divided (66.7%) during the initial 24 h co-culture period with parental embryos. Half of the biopsied material from an individual embryo was then transferred onto HFF PD14 and the other half was transferred onto HFF PD6. Culture conditions were as described in Example 1. By day 3, all blastomeres that divided during co-culture with a parental embryo, attached to feeder cells, regardless of HFF PD. At day 5, initial outgrowth was detected in all cultures having HFF PD6 (100%), and in 3 out of 4 cultures having HFF PD14 (75%). Growing hESC colonies were observed in 2 out of 4 cultures (50%) on HFF PD6 several days later. No hESC colony growth was seen on HFF PD14. Both newly derived lines had typical hESC morphology, and in a series of assays demonstrated their pluripotency. One line has retained a stable normal female karyotype (46,XX), whereas the other line has retained a stable normal male karyotype (46,XY) over an extended period of culture. Analysis of fifteen STR loci in one of the lines indicated an allele frequency of 1.63×10⁻¹⁸, meaning that less than 1 in 6.14×10¹⁷ individuals of unspecified race could be expected to match its DNA profile. The same analysis in the other line indicated an allele frequency of 3.29×10⁻¹⁹, meaning that less than 1 in 3.04×10¹⁸ individuals of unspecified race could be expected to match its DNA profile.

This set of experiments demonstrated that derivation of hESC lines from biopsied blastomeres on HFF could be improved by use of earlier passage feeder cells. Thus, even though passage number had no dramatic effect on attachment (100% for PD6 vs. 92.3% for PD14) earlier passage number clearly favors formation of an initial outgrowth (100% for PD6 vs. 66.7% for PD14). Superiority of earlier passage feeder cells for hESC culture was even more pronounced in terms of the effect on derivation where the efficiency of derivation was 50% for PD6 as compared to 12.5% for PD14.

### Example 3

### Simplification of hESC Derivation Method

This experiment aimed to determine, using genetically identical material, whether co-culture with parental embryo is essential for derivation of hESC line from biopsied blastomeres.

Two grade 2 surplus frozen human cleavage stage embryos donated by one consenting couple were thawed and incubated in Quinn's cleavage medium for a minimum of 3 h at standard culture conditions. As in Example 2, genetically identical samples were obtained by extracting two blastomeres per embryo. Half of the biopsied material from each individual embryo was then transferred into another drop of Quinn's cleavage medium, and the other half remained for 24 h in the same drop with the parental embryo. From one of the embryos, both individually cultured and co-cultured blastomeres divided within the first 24 h, whereas from the second embryo neither one did. Two blastomere-derived aggregates from the first embryo as well as undivided blastomeres from the second embryo were transferred into individual drops with PD6 HFF. Culture conditions were as described in the Examples 1 and 2. Neither blastomere from the second embryo progressed further, however both blastomere-derived aggregates from the first embryo attached and formed an initial outgrowth by day 5. About a week later, outgrowth from the blastomere cultured in the absence of the parental embryo gave rise to an hESC line. This newly derived line had typical hESC morphology, and in a series of assays demonstrated pluripotency. It has retained a stable normal female karyotype (46,XX) over an extended period of culture. Analysis of fifteen STR loci in this line indicated an allele frequency of 3.43×10⁻¹⁹, meaning that less than 1 in 2.91×10¹⁸ individuals of unspecified race could be expected to match the DNA profile for this hESC line.

Co-culture of biopsied blastomeres with parental embryos therefore is not essential to generate hESC lines from blastomeres. This finding is important since it suggests that parental embryos can be maintained in optimal conditions thereby increasing their likelihood of blastocyst development.

### Example 4

### Serum-Free and Low Oxygen hESC Derivation

In a separate set of experiments, two blastomeres were extracted from each of three embryos and each was tested for its ability to give rise to an hESC line. For each blastomere pair (i.e., two blastomeres extracted from a single embryo), one blastomere was cultured in the absence of serum and in low oxygen (8%) while the other blastomere was cultured in the absence of serum and in high oxygen (about 20%). Low and high oxygen culture conditions both employed human foreskin fibroblasts as feeder cells. For each of the blastomere pairs, the blastomere cultured in the absence of serum and in low oxygen generated an hESC line while the blastomere cultured in the absence of serum and in high oxygen did not. Thus, in these experiments, low oxygen replaced any requirement for serum.

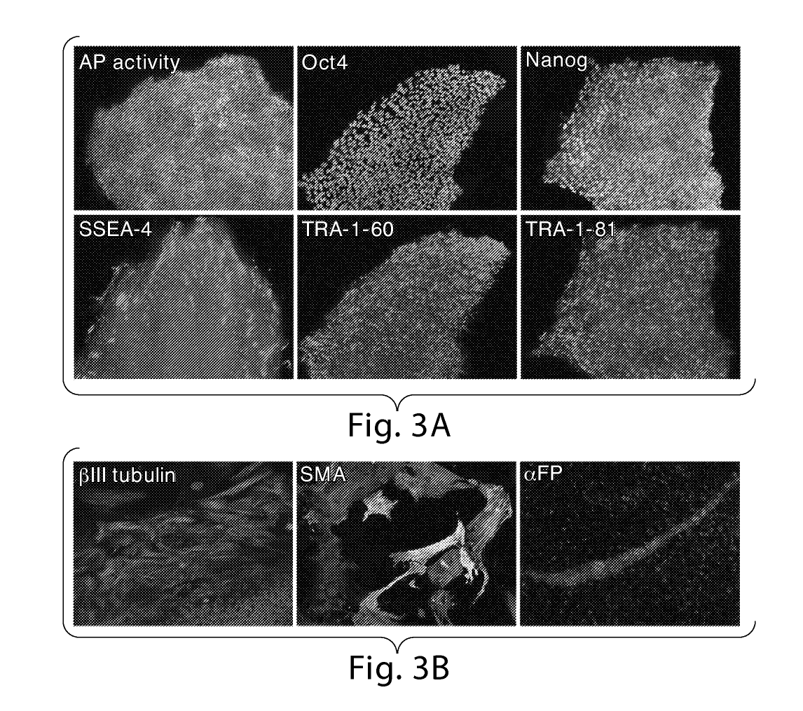

All three hESC lines have a normal karyotype (i.e., two are 46 X,Y and one is 46 X,X) and unique DNA fingerprint. This latter characteristic is evidence that none of the lines is the result of contamination by other pre-existing cell lines. The hESC lines generated exhibit normal growth rate and morphology and express markers of pluripotency that are characteristic for undifferentiated hESC including expression of transcription factors Oct-3/-4 and Nanog, and cell surface markers SSEA-4, Tra 1-1-60 and Tra-1-81, and alkaline phosphatase activity (FIG. 3A). In addition, the lines each exhibit ectodermal, mesodermal and endodermal differentiative potential as evidenced by immunostaining for beta III tubulin (bIII tubulin), alpha smooth muscle actin (SMA) and alpha feto protein (aFP), respectively (FIG. 3B). FIGS. 3A and 3B show results with one of these cell lines and are representative of the results found with the remaining two lines.

### Example 5

### Generation of Biopsied Blastomere hESC Line in Xeno-Free Medium on Human Feeders

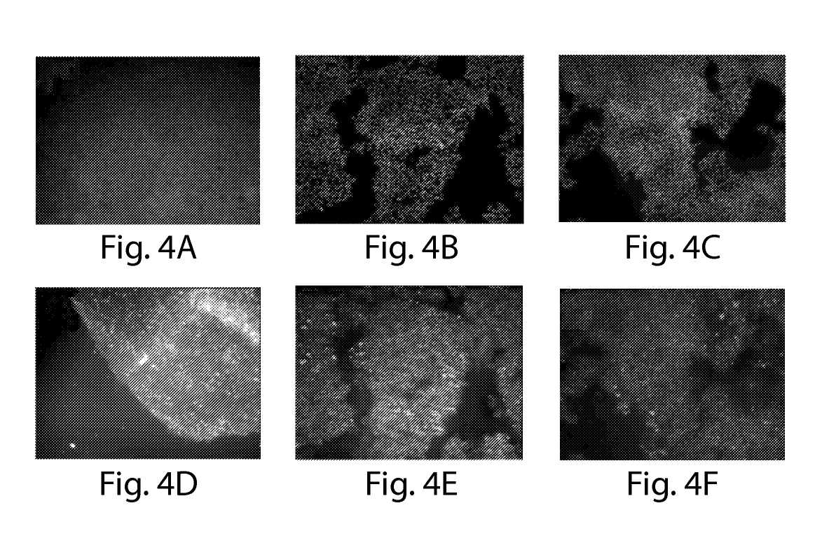

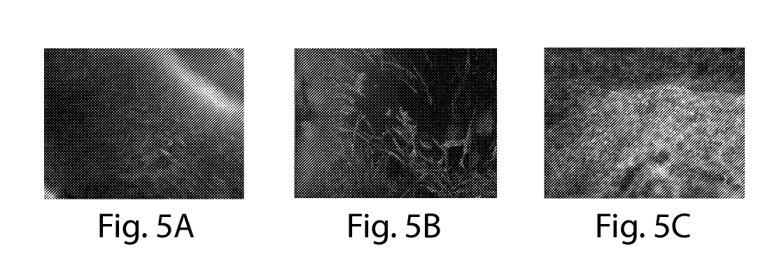

Embryos donated by a consenting couples were thawed and incubated in Quinn's cleavage medium for a minimum of 3 hours at standard culture conditions. Blastomeres were removed using an established biopsy procedure (Chung 2008). Biopsied blastomeres were cultured in separate drops of Quinn's cleavage medium for 24 hours. After 24 hours of culture, blastomeres were transferred onto feeder drops containing Quinn's blastocyst medium and 10 μg/ml human laminin (50 μl) and cultured at 37° C. under 5% CO₂, 8% O₂ and humidified air for 72 hours without disturbance. After 72 hours, cultures were assessed for blastomere attachment to feeders and an initial cell outgrowth by day 5. Starting at day 3, medium in drops containing attached blastomeres was refreshed every day by replacing ⅓ of the volume with Quinn's blastocyst medium supplemented with 10 μg/ml laminin, 10 ng/ml LIF, and 50 ng/ml bFGF. From day 5, Quinn's blastocyst medium was replaced with commercially available Xeno free hESC medium from Invitrogen enriched with 10 μg/ml human laminin, 10 ng/ml LIF and 50 ng/ml bFGF. Most of trophoblast outgrowth died on day 6 and 7. On day 9, initial hESC colonies could be detected. Each colony was dissected within the same drop two days later and again at day 14. After the second dissection, small hESC clumps were transferred into 4-well dish with new HFF PD14 feeders containing Xeno free hESC medium from Invitrogen enriched with 10 μg/ml human laminin, 10 ng/ml LIF and 25 ng/ml bFGF. The next day, attached clumps of hESC cultured in Xeno free hESC medium were observed. The newly derived cell line had a typical morphology of hESC. Enzymatic assay (alkaline phosphatase /AP/activity) and immunostaining (Oct-3/4, Nanog, SSEA-4, TRA-1-60, and TRA-1-80) were used to confirm expression of pluripotency markers (FIGS. 4A-F). Differentiation potential into all three embryonic germ layer derivatives was confirmed by immunostaining (FIGS. 5A-C). The hESC line has shown and retained stable normal female karyotype (46,XX) over an extended period of culture.

### EQUIVALENTS

It should be understood that the preceding is merely a detailed description of certain embodiments. It therefore should be apparent to those of ordinary skill in the art that various modifications and equivalents can be made without departing from the spirit and scope of the invention, and with no more than routine experimentation. All references, patents and patent applications that are recited in this application are incorporated by reference herein in their entirety.

What is claimed is:

1. A method for producing human embryonic stem cells comprising
culturing a human blastomere and/or its progeny in the presence of human adult feeder cells and in the absence of other cells for a time sufficient to generate embryonic stem cells, and
isolating human embryonic stem cells.

2. The method of claim 1, wherein the human adult feeder cells are human foreskin fibroblast cells.

3. The method of claim 1, wherein the human adult feeder cells are present in a density of about 2-3×10⁵ cells/ml.

4. The method of claim 1, wherein the human adult feeder cells are irradiated.

5. The method of claim 1, wherein the human adult feeder cells are early passage feeder cells.

6. The method of claim 1, wherein the human adult feeder cells have been passaged 4-8 times.

7. The method of claim 1, wherein the human blastomere is not cultured in the presence of its parental embryo prior to culture with the human adult feeder cells.

8. The method of claim 1, wherein the human embryonic stem cells are isolated at about 10-15 days of culture.

9. The method of claim 1, wherein the human blastomere is cultured in the absence of animal serum.

10. The method of claim 1, wherein the human blastomere is cultured in the presence of animal serum for 4-10 days.

11. The method of claim 1, wherein the human blastomere is cultured in low oxygen.

12. A method for improving the efficiency of human embryonic stem cell production from blastomeres comprising
culturing a human blastomere and its progeny in the presence of human foreskin fibroblast cells and in the absence of other cells for a time sufficient to generate embryonic stem cells, wherein the human blastomere and human foreskin fibroblast cells are present in a ratio of about 1:10000 to about 1:15000, and
isolating human embryonic stem cells.

13. The method of claim 12, wherein the human foreskin fibroblast cells are early passage feeder cells.

14. The method of claim 12, wherein the human foreskin fibroblast cells have been passaged 4-8 times.

15. The method of claim 12, wherein the human foreskin fibroblast cells are irradiated.

16. The method of claim 12, wherein the human blastomere is not cultured in the presence of its parental embryo prior to culture with the human foreskin fibroblast cells.

17. The method of claim 12, wherein the human embryonic stem cells are isolated at about 10-15 days of culture.

18-23. (canceled)

24. A method for producing human embryonic stem cells comprising
in a first culturing step, culturing a human blastomere from a human embryo in the absence of feeder cells,
in a second culturing step, culturing the human blastomere and its progeny in the presence of human adult feeder cells and in the absence of other cells,
in a third culturing step, culturing the human blastomere and its progeny in the presence of human adult feeder cells and animal serum,
in a fourth culturing step, culturing the human blastomere and its progeny in the absence of animal serum, and
isolating embryonic stem cells.

25-37. (canceled)

38. A method for producing human embryonic stem cells comprising
culturing a human blastomere and/or its progeny in the presence of human foreskin fibroblasts, in the absence of other cells, in the absence of serum, and in low oxygen for a time sufficient to generate embryonic stem cells, and
isolating human embryonic stem cells.

39-40. (canceled)

41. A method for improving efficacy of human embryonic stem cell derivation in the absence of serum, comprising
culturing a human blastomere and/or its progeny in the absence of serum and in low oxygen for a time sufficient to generate embryonic stem cells, and
isolating human embryonic stem cells.

42-45. (canceled)
